(19) United States
(12) Patent Application Publication
(10) Pub. No.: US 2025/0009763 A1
(43) Pub. Date: Jan. 9, 2025

(54) METHODS FOR EXTENDING LIFESPAN AND/OR DELAYING AGING

(71) Applicant: Chang Gung University, Taoyuan City (TW)

(72) Inventors: Chin-Chuan CHEN, Taoyuan City (TW); Yann-Lii LEU, Taoyuan City (TW); Chi-Yuan CHEN, Taoyuan City (TW); Tong-Hong WANG, Taoyuan City (TW); Shu-Fang CHENG, Taoyuan City (TW)

(73) Assignee: Chang Gung University, Taoyuan City (TW)

(21) Appl. No.: 18/219,105

(22) Filed: Jul. 7, 2023

(51) Int. Cl. A61K 31/575; A61K 45/06; A61P 43/00

(52) U.S. Cl. CPC A61K 31/575; A61K 45/06; A61P 43/00

(57) ABSTRACT

Disclosed herein is related to a method for extending lifespan and/or delaying aging in a subject; the method comprises administering to the subject with an effective amount of (24S)-3β-hydroxy-5α-stigmastan-6-one, wherein the delaying aging comprises increasing vitality, muscle strength or motor coordination, insulin sensitivity, or basal metabolic rate; and/or reducing muscle weakness, loss of balance, hair graying, kyphosis, or hyperglycemia. Also encompassed in the present disclosure are methods for promoting weight loss or treating type 2 diabetes mellitus in a subject, in which the methods comprise administering to the subject an effective amount of (24S)-3β-hydroxy-5α-stigmastan-6-one.

### BACKGROUND OF THE INVENTION

### 1. Field of the Invention

The present disclosure in general relates to the field of biological pharmaceuticals. More particularly, the present disclosure relates to (24S)-3β-hydroxy-5α-stigmastan-6-one (PN6) and uses thereof for promoting longevity and/or treating conditions or symptoms associated with senescence.

### 2. Description of Related Art

Aging is a natural biological process that affects all living organisms, including humans. It is a complex process that affects all cells, tissues, and organs in the body and is characterized by a progressive decline in physiological functions over time. At the cellular level, the accumulation of senescent cells in tissues and organs may contribute to aging, leading to a deterioration of biological functions and age-related health problems. Dysregulated molecular mechanisms of aging, such as loss of DNA repair capacity, chromosome instability, telomere erosion, loss of proteostasis, deregulated nutrient-sensing, free radical generation, stem cell exhaustion, altered intercellular communication, and the like, may trigger the formation of senescent cells that further develop a senescence-associated secretory phenotype (SASP) contributing to the senescence process in surrounding tissues. As senescent cells accumulate over time, they lead to a deterioration of organ function, increasing the risk of age-related diseases and ultimately mortality. Thus, cellular senescence has a significant impact on an individual's health. The development of anti-aging drugs could promote healthy aging and treat aging-related diseases; nonetheless, only a few compounds with anti-aging activities have been developed to date.

On the other hand, several pharmacopoeias of traditional Chinese medicine (TCM), such as the Compendium of Materia Medica, Qianjinyaofang, Shennong Materia Medica, and Huangdi Neijing, record that many TCMs possess beneficial medical effects. Among them, plumula nelumbinis (PN) is of particular interest, as it displays numerous beneficial biological activities, including antioxidant, anti-hypertensive, anti-asthmatic, anti-restenotic, anti-neurodegenerative, anti-diabetic, anti-autoimmune, anti-arrhythmic, anti-Alzheimer's, anti-cancer, hepatoprotective, anti-inflammatory, anti-aging, and other such activities. Nevertheless, as the key compounds that may contribute to the potential anti-aging activities of PN have not been identified, the activities associated with them have yet to be validated. Recently, advances have been made in technologies for compound purification and in establishment of an in vitro screening platform for anti-aging drugs (e.g., the mother enrichment program (MEP)), which enables the uncovering of the potential anti-aging ingredients from PN.

In view of the foregoing, there exists a need of identifying active substances with anti-aging properties from PN, in which the active substances may serve as candidate compounds for developing medicaments suitable for ameliorating cellular senescence, and/or treating aging-related diseases.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to a method for extending lifespan and/or delaying aging in a subject, comprising administering to the subject an effective amount of (24S)-3β-hydroxy-5α-stigmastan-6-one, wherein the delaying aging comprises increasing vitality, muscle strength or motor coordination, insulin sensitivity, or basal metabolic rate; and/or reducing muscle weakness, loss of balance, hair graying, kyphosis, or hyperglycemia.

According to some embodiments of the present disclosure, the (24S)-3β-hydroxy-5α-stigmastan-6-one has been shown to extend lifespan and/or to delay aging in a subject, partly through its activation of mammalian SIRT1; as such, the (24S)-3β-hydroxy-5α-stigmastan-6-one may be an activator of mammalian SIRT1. Alternatively or in addition, the (24S)-3β-hydroxy-5α-stigmastan-6-one has been shown to increase the NAD⁺ content; as such, the (24S)-3β-hydroxy-5α-stigmastan-6-one may be an activator of NAD⁺.

According to some embodiments of the present disclosure, the (24S)-3β-hydroxy-5α-stigmastan-6-one is administered to the subject in the amount of about 1-10 mg/kg.

Preferably, the subject treatable by the present method is a mammal, for example, a human, a mouse, a rat, a guinea pig, a hamster, a monkey, a swine, a dog, a cat, a horse, a sheep, a goat, a cow, and a rabbit. According to one preferred embodiment of the present disclosure, the subject is a human.

Another aspect of the present disclosure is directed to a method for promoting weight loss or treating type 2 diabetes mellitus in a subject comprising administering to the subject an effective amount of (24S)-3β-hydroxy-5α-stigmastan-6-one.

According to some embodiments of the present disclosure, the (24S)-3β-hydroxy-5α-stigmastan-6-one is administered to the subject in the amount of about 1-10 mg/kg.

In the case when treating type 2 diabetes mellitus, the method further comprises administering an antidiabetic agent to the subject. According to the embodiments of the present disclosure, the antidiabetic agent is selected from the group consisting of insulin, metformin, phenformin, buformin, rosiglitazone, pioglitazone, troglitazone, tolimidone, bromocriptine, colesevelam, glitazone, gliptin, tolbutamide, acetohexamide, tolazamide, chlorpropamide, glipizide, glyburide, glibenclamide, glimepiride, gliclazide, glyclopyramide, gliquidone, repaglinide, nateglinide, miglitol, acarbose, voglibose, exenatide, liraglutide, taspoglutide, lixisenatide, semaglutide, dulaglutide, albiglutide, vildagliptin, sitagliptin, saxagliptin, linagliptin, alogliptin, septagliptin, teneligliptin, gemigliptin, dapagliflozin, canagliflozin, empagliflozin, and remogliflozin.

Preferably, the subject treatable by the present method is a human.

Many of the attendant features and advantages of the present disclosure will becomes better understood with reference to the following detailed description considered in connection with the accompanying drawings.

### BRIEF DESCRIPTION OF THE DRAWINGS

These and other features, aspects and advantages of the present invention will become better understood with reference to the following description, appended claims and the accompanying drawings, where:

FIGS. 1A-1F depict the effect of (24S)-3β-hydroxy-5α-stigmastan-6-one (PN6) on extending the replicative lifespan (RLS) of yeast (Saccharomyces cerevisiae haploid MEP strain ZHY2) in accordance with the embodiments of the present disclosure. FIGS. 1A-1D are the results of the mother enrichment program (MEP) assay for assessing the RLS of the yeasts under the indicated concentrations of PN6, in which FIGS. 1A and 1C depict the viability rate (%) of the yeasts over time, and FIGS. 1B and 1D are the scatter plots respectively summarizing the results of FIGS. 1A and 1C; the yeast strain with fob1 deletion (fob1Δ) was used as a positive control for the MEP assay (n=4). FIGS. 1E-1F are the results of the micromanipulation assay for further confirmation of the RLS of the yeasts under the indicated treatments, in which FIG. 1E depicts the RLS results of the yeasts under the indicated PN6 concentrations (n=104), and FIG. 1F depicts the RLS results of the yeasts under the treatments of PN6 (25 μM) or β-sitosterol (25 μM; a phytosterol, having a chemical structure similar to PN6, served as a control for chemical structure) (n=110); the yeast strain with fob1 deletion (fob1Δ) was used as a positive control for RLS; the number within parentheses represents the average lifespan (in generations).

FIGS. 2A-2K demonstrate the effect of (24S)-3β-hydroxy-5α-stigmastan-6-one (PN6) on the extension of the RLS of yeast via SIR2 pathway. FIG. 2A shows the assessment results of the RLS of the yeast strains: the wild-type (WT) strain and the sir2 and fob1 double deletion (sir2Δfob1Δ) strain, in the presence or absence of PN6 (25 μM); the number within parentheses represents the average lifespan (in generations) (n=128-129). FIG. 2B illustrates the effect of PN6 or caloric restriction (CR) 0.1% (reducing glucose to 0.1% (CR 0.1%), served as a positive control) on the level of intracellular NAD⁺ (n=4). FIGS. 2C-2J are the results depicting the expression of acetyl-p53 (p53ac, p53 acetylation at K382) in U2OS (FIGS. 2C-2F) and IMR90 (FIGS. 2G-2J) under the indicated treatments, in which FIGS. 2C, 2E, 2G, and 2I show the western blotting results, and FIGS. 2D, 2F, 2H, and 2J present corresponding bar diagrams respectively quantifying the results from FIGS. 2C, 2E, 2G, and 2I; selisistat (Ex527): an inhibitor for yeast SIR2 or mammalian SIRT1 (n=3). FIG. 2K is the result depicting the activity of yeast SIR2 in response to treatment with PN6 at the indicated concentrations; *, p<0.05; **, p<0.01; ****, p<0.001.

FIGS. 3A-3C depict the effect of (24S)-3β-hydroxy-5α-stigmastan-6-one (PN6) on cellular senescence in accordance with the embodiments of the present disclosure.

FIG. 3A is the result for assessment of the population doubling level (PDL) of human umbilical vein endothelial cells (HUVECs) under the indicated treatments. FIG. 3B is the western blotting result depicting the expression of p21 in HUVECs at PDL4 or PDL11 under the indicated concentrations of PN6. FIG. 3C is the scatter plot result depicting the ratio (%) of senescence-associated (SA)-β-gal-positive HUVECs at PDL4 or PDL11 under the indicated concentrations of PN6.

FIGS. 4A-4O depict the effect of (24S)-3β-hydroxy-5α-stigmastan-6-one (PN6) on the physical functions of the aged mice fed a standard rodent chow with or without 50 mg/kg of PN6 (designated as standard dietary (SD) and PN6). FIGS. 4A-4C are the results of the survival rate (FIG. 4A), the body weight (FIG. 4B), and the food intake (FIG. 4C) of the aged mice in the indicated treatment groups (SD, n=27; PN6, n=10). FIG. 4D is the result of the rearing behavior of the aged mice in the indicated treatment groups during the mid-term (at 72 weeks of age) or long-term (at 111 weeks of age) (mid-term: SD, n=16; PN6, n=8; long-term: SD, n=12; PN6, n=8). FIGS. 4E-4G are the results of the grip strength of the forelimbs (FIG. 4E) or the hindlimbs (FIG. 4F), and of all the four limes (FIG. 4G) of the aged mice in the indicated treatment groups during the mid-term (at 87 weeks of age) or long-term (at 111 weeks of age) (mid-term: SD, n=30; PN6, n=8; long-term: SD, n=23; PN6, n=8). FIGS. 4H-4I are the results of the latency to falling in the rotarod tests at constant speed (FIG. 4H) or accelerating speed (FIG. 4I) of the aged mice in the indicated treatment groups during the mid-term (at 88 weeks of age) or long-term (at 112 weeks of age) (mid-term: SD, n=30; PN6, n=8; long-term: SD, n=22; PN6, n=8). FIG. 4J is the result of the insulin tolerance test of the aged mice in the indicated treatment groups, in which the blood glucose was measured at the specified time points after insulin administration; *, p<0.05 (SD, n=24; PN6, n=8). FIG. 4K is the scatter plot summarizing the result of FIG. 4J. FIGS. 4L-4O depict the outcomes of the metabolic tests of the aged mice in the indicated treatment groups; these tests measured oxygen (O₂) consumption (FIG. 4L) and carbon dioxide (CO₂) elimination (FIG. 4M), which were used to calculate the respiratory quotient (FIG. 4N) and energy expenditure (FIG. 4O) (SD, n=24; PN6, n=8).

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a,” “an,” and “the” include the plural reference unless the context clearly dictates otherwise. Also, as used herein and in the claims, the terms “at least one” and “one or more” have the same meaning and include one, two, three, or more. The practice of the present invention will employ, unless otherwise indicated, conventional techniques of biochemistry, molecular biology, and the like, which are within the skill of the art. Such techniques are explained fully in the literature.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “aging” as used herein refers to an individual who exhibits symptoms or clinical manifestations related to physical deterioration, diseases, or disorders typically associated with the aging process, at a particular age (i.e., senescence). Such symptoms or clinical manifestations associated with senescence may result from a loss of energy or vitality, muscle dysfunction such as reduced strength or increased weakness, impaired balance ability such as reduced motor coordination or loss of balance, metabolic degeneration such as decreased insulin sensitivity (thus resulting in hyperglycemia) or a reduced basal metabolic rate, or visible changes in appearance such as graying hair or kyphosis. The symptoms or clinical manifestations associated with senescence as described herein may be at least in part due to cellular senescence, and may further evolve into senescence-associated diseases, such as hypertension, sarcopenia, arthritis (e.g., degenerative arthritis, rheumatoid arthritis, metabolic arthritis, infectious arthritis, or spondylitis ankylosans), osteoporosis, or a neurodegenerative disease (e.g., Alzheimer's disease, vascular dementia, dementia with Lewy bodies, frontotemporal lobar degeneration, Parkinson's disease, Huntington's Disease, amyotrophic lateral sclerosis, multiple sclerosis, spinal muscular atrophy), which may be treatable with the present method.

A “decreased” or “reduced” level or amount is typically a “statistically significant” level or amount, and may include, for example, a 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease (including all integers and ranges in between) relative to a control. An decreased or reduced level 10 or amount may also include a 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 1,000-fold, 10,000-fold, or greater than 10,000-fold decrease (including all integers and ranges in between) relative to a control. Other examples of comparisons and “statistically significant” levels or amounts are described herein. “Decrease,” as used herein, can refer to “inhibit,” “reduce,” “curb,” “abate,” “diminish,” “lessen,” or “lower.”

A “increased” or “enhanced” level or amount is typically a “statistically significant” level or amount, and may include, for example, a 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% increase (including all integers and ranges in between) relative to a control. An increased or enhanced level or amount may also include a 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 1,000-fold, 10,000-fold, or greater than 10,000-fold increase (including all integers and ranges in between) relative to a control. Other examples of comparisons and “statistically significant” levels or amounts are described herein. “Increase,” as used herein, can refer to “agonize,” “enhance,” “inflate,” “escalate,” expand,” “augment,” “enlarge,” or “raise.”

The term “statistically significant” refers to the result was unlikely to have occurred by chance. Statistical significance may be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less. The term “significant” encompasses and includes the term “statistically significant.”

The term “subject” or “patient” refers to an animal including the human species that is treatable with the pharmaceutical composition and/or the method of the present disclosure. The term “subject” or “patient” intended to refer to both the male and female gender unless one gender is specifically indicated. Accordingly, the term “subject” or “patient” comprises any mammal which may benefit from administration of (24S)-3β-hydroxy-5α-stigmastan-6-one. Examples of a “subject” or “patient” include, but are not limited to, a human, a rat, a mouse, a guinea pig, a monkey, a pig, a goat, a cow, a horse, a dog, a cat, a bird, and a fowl. In an exemplary embodiment, the patient is a human.

The terms “treatment” and “treating” as used herein may refer to a curative or palliative measure. In particular, the term “treating” as used herein refers to the application or administration of the present (24S)-3β-hydroxy-5α-stigmastan-6-one or a pharmaceutical composition comprising the same to a subject, who has a symptom or a disease associated with senescence, or a disease or a disorder secondary to the symptom or the disease associated with senescence, with the purpose to partially or completely alleviate, ameliorate, relieve, delay onset of, inhibit progression of, reduce severity of, and/or reduce incidence of one or more symptoms or features associated with senescence.

The term “administered,” “administering” or “administration” are used interchangeably herein to refer either directly administering the present (24S)-3β-hydroxy-5α-stigmastan-6-one, or the present pharmaceutical composition comprising (24S)-3β-hydroxy-5α-stigmastan-6-one.

The term “an effective amount” as used herein refers to an amount effective, at dosages, and for periods of time necessary, to achieve the desired therapeutically desired result with respect to the treatment of symptoms or diseases associated with senescence in a subject. For therapeutic purposes, the effective amount is also one in which any toxic or detrimental effects of the component are outweighed by the therapeutically beneficial effects. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/kg). Alternatively, the effective amount can be expressed in the concentration of the active component (e.g., the present (24S)-3β-hydroxy-5α-stigmastan-6-one), such as molar concentration, mass concentration, volume concentration, molality, mole fraction, mass fraction and mixing ratio. Persons having ordinary skills could calculate the human equivalent dose (HED) for the medicament (such as the present (24S)-3β-hydroxy-5α-stigmastan-6-one) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “pharmaceutical composition” refers to a preparation which is in such form as to permit the biological activity of an active ingredient contained therein to be effective, and which contains no additional components which are unacceptably toxic to a subject to which the composition would be administered. In general, the amount of active compounds (i.e., (24S)-3β-hydroxy-5α-stigmastan-6-one) is present in the pharmaceutical composition at a level of about 0.01% to 99% by weight; preferably, at a level of at least 0.1% by weight; more preferably, at a level of at least 1% by weight; even more preferably, at a level of at least 5% by weight; yet even more preferably, at a level of at least 10% by weight; still yet even more preferably, at a level of at least 25% by weight, based on the total weight of the pharmaceutical composition. For the clinical use of the present invention, the present pharmaceutical composition is formulated into formulations suitable for the intended route of administration.

The phrase “pharmaceutically acceptable excipient” as used herein means a pharmaceutically acceptable material, composition or vehicle, such as a liquid or solid filler, diluent, carrier, solvent or encapsulating material, involved in carrying or transporting the subject agents from one organ, or portion of the body, to another organ, or portion of the body. Each excipient must be “acceptable” in the sense of being compatible with the other ingredients of the formulation. The pharmaceutical formulation contains a compound of the invention in combination with one or more pharmaceutically acceptable ingredients. The excipient can be in the form of a solid, semi-solid or liquid diluent, cream or a capsule. These pharmaceutical preparations are a further object of the invention.

### II. Description of the Invention

The present disclosure is based, at least in part, on the discovery that (24S)-3β-hydroxy-5α-stigmastan-6-one can enhance physical functions of a live subject, such as promotion of vitality, muscle strength, and balance, blood sugar level regulation, metabolism enhancement, maintenance of a youthful appearance, etc.; accordingly, (24S)-3β-hydroxy-5α-stigmastan-6-one may contribute to individual rejuvenation and potentially delay the aging process, and it may also be used in promoting weight loss or treating type 2 diabetes mellitus.

### 1. The Present Composition

Accordingly, the first aspect of the present disclosure is to provide a composition for treating symptoms or diseases associated with senescence as described herein. The composition comprises (24S)-3β-hydroxy-5α-stigmastan-6-one, and a carrier; the (24S)-3β-hydroxy-5α-stigmastan-6-one has the chemical structure of

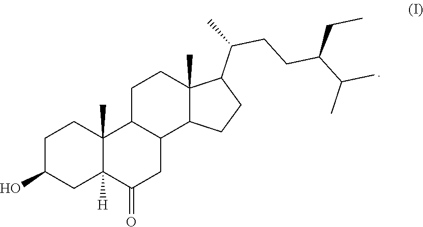 [SMILES: CC[C@H](CC[C@@H](C)C1CCC2C3CC(=O)[C@H]4C[C@@H](O)CC[C@]4(C)C3CC[C@@]21C)C(C)C]

The (24S)-3β-hydroxy-5α-stigmastan-6-one comprised in the present composition may be obtained by extracting from natural herbs or by chemical synthesis. Said natural herbs include, but are not limited to, plumula nelumbinis (PN) derived from Nymphaea alba, Nymphaea odorata, Nymphaea rubra, Nuphar lutea, Nuphar japonica, Lotus berthelotii, Lotus corniculatus, Victoria amazonica, Victoria cruziana, Nelumbo nucifera, Nelumbo lutea, Euryale ferox, Cabomba caroliniana, Brasenia schreberi, Barclaya longifolia, Victoria regia, Nymphaea tetragona, Nuphar advena, Nuphar microphylla, Nymphaea candida, Nymphaea capensis, Nymphaea gigantea, Nymphaea lotus, Nymphaea micrantha, Nymphaea mexicana, Nymphaea minuta, Nymphaea nouchali, Nymphaea pubescens, Nymphaea stellata, and Nymphaea thermarum. Preferably, the present (24S)-3β-hydroxy-5α-stigmastan-6-one may be extracted from PN of Nelumbo nucifera, via any extraction methods known in the art. Preferably, the present (24S)-3β-hydroxy-5α-stigmastan-6-one may be obtained by mixing any of the herbs described above with an extractant, such as acetone, dichloromethane, ethanol (EtOH), ethyl acetate (EtOAc), n-hexane, water, and etc., for a sufficient period of time to produce a crude extract.

Additionally or alternatively, the crude extract may be further purified to give substantially pure (24S)-3β-hydroxy-5α-stigmastan-6-one. Generally, the crude extract is purified by column chromatography (e.g., high performance liquid chromatography (HPLC), silica gel chromatography), thin-layer chromatography, recrystallization, or a combination thereof. In one working example, the crude extract is subjected to silica gel chromatography to give substantially pure (24S)-3β-hydroxy-5α-stigmastan-6-one.

The purified compound is then identified by methods including, but are not limited to, chemical analysis (e.g., titration analysis, gravimetric analysis, or micro-spectroscopy), spectroscopic analysis (e.g., optical rotation spectroscopy, atomic spectroscopy, or molecular spectroscopy), mass spectrometry, nuclear magnetic resonance (NMR) proton spectroscopy, chromatography (e.g., column chromatography, ion-exchange chromatography, gel chromatography, affinity chromatography, HPLC, or thin-layer chromatography), infrared spectroscopy, or a combination thereof (e.g., liquid chromatography-mass spectrometry (LC-MS)). In one working example, the present (24S)-3β-hydroxy-5α-stigmastan-6-one are identified by NMR proton spectroscopy.

The composition comprising the (24S)-3β-hydroxy-5α-stigmastan-6-one may be utilized in the form of a pharmaceutical (including a nutritional supplement), a cosmetic, or a health functional food according to its purposes.

According to some embodiments of the present disclosure, the composition is used as the pharmaceuticals (or the nutritional supplement), in such case, the pharmaceutical composition may further comprise suitable carriers, excipients, and diluents commonly used in the manufacture of pharmaceutical compositions. In addition, the pharmaceutical composition may be formulated and used in the form of oral dosage forms such as powders, granules, tablets, capsules, suspensions, emulsions, syrups, aerosols, external preparations, suppositories; and sterile injectable solutions according to a conventional method. Carriers, excipients, and diluents that may be included in the pharmaceutical composition comprise lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil. In the case of formulation, it is prepared using excipients or diluents such as fillers, extenders, binders, wetting agents, disintegrating agents, and surfactants. Solid preparations for oral administration include tablets, pills, powders, granules, capsules, etc. These solid preparations include at least one excipient in the preparation, for example, starch, calcium carbonate, sucrose, lactose, gelatin, and the like. In addition, lubricants such as magnesium stearate or talc are used in addition to simple excipients. Liquid preparations for oral use include suspending agents, solutions, emulsions, syrups, etc. In addition to water and liquid paraffin, which are commonly used as simple diluents, various excipients, such as wetting agents, sweeteners, fragrances, and preservatives, may be included in the preparation. Formulations for parenteral administration include sterile aqueous solutions, non-aqueous solvents, suspensions, emulsions, lyophilized preparations, and suppositories. Non-aqueous solvents and suspensions may include propylene glycol, polyethylene glycol, vegetable oils such as olive oil, and injectable esters such as ethyl oleate. As a base for suppositories, witepsol, macrogol, Tween-61, cacao butter, laurin butter, and glycerogelatin may be used.

According to some embodiments of the present disclosure, the composition is used as the cosmetics, in such case, components commonly used in the cosmetic composition may be included in addition to the active ingredient, for example, a stabilizer, a solubilizing agent, a common auxiliary agent such as vitamins, pigments and fragrances, and a carrier. In addition, the cosmetic composition may be prepared in any formulations conventionally prepared in the art, such as solutions, suspensions, emulsions, pastes, gels, creams, lotions, powders, soaps, surfactant-containing cleansings, oils, powders. It may be formulated as a foundation, emulsion foundation, wax foundation, spray, etc., but is not limited thereto. In such cases when the formulation is a paste, cream or gel, animal oil, vegetable oil, wax, paraffin, starch, tragacanth, cellulose derivative, polyethylene glycol, silicone, bentonite, silica, talc, zinc oxide, etc. may be used as carrier components. In addition, in the case when the formulation is a powder or a spray, lactose, talc, silica, aluminum hydroxide, calcium silicate, or polyamide powder may be used as a carrier component, and especially in the case of a spray, additionally propellants such as chlorofluorohydrocarbon, propane/butane, and dimethyl ether may be used. In addition, when the formulation is a solution or an emulsion, a solvent, a solubilizing agent or an emulsifying agent is used as a carrier component, such as water, ethanol, isopropanol, ethyl carbonate, ethyl acetate, benzyl alcohol, benzyl benzoate, propylene glycol, fatty acid esters of 3-butyl glycol oil, glycerol aliphatic esters, polyethylene glycol, or sorbitan may be used. Also, when the formulation is a suspension, liquid diluents such as water, ethanol, and propylene glycol, ethoxylated isostearyl alcohol, suspensions such as polyoxyethylene sorbitol esters and polyoxyethylene sorbitan esters, microcrystalline cellulose, aluminum metahydroxide, bentonite, agar, trakant, and the like may be used as carrier components. In addition, when the above formulation is surfactant-containing cleansings, aliphatic alcohol sulfate, aliphatic alcohol ether sulfate, sulfosuccinic acid monoester, isethionate, imidazolinium derivatives, methyl taurate, sarcosinate, fatty acid amide ether sulfates, alkylamidobetaines, aliphatic alcohols, fatty acid glycerides, fatty acid diethanolamides, vegetable oils, lanolin derivatives, ethoxylated glycerol fatty acid esters, and the like may be used as the carrier component.

According to some embodiments of the present disclosure, the composition is utilized as the health functional food, ingredients commonly used in the health functional food may be included in addition to the active ingredient, for example, citric acid, oligosaccharide, taurine, fruit concentrate, etc. may be included when the composition is manufactured as a health functional beverage.

### 2. Uses of the Present Composition

Another aspect of the present disclosure pertains to a method for extending lifespan and/or delaying aging in a subject; said delaying aging comprises increasing vitality, muscle strength or motor coordination, insulin sensitivity, or basal metabolic rate; and/or reducing muscle weakness, loss of balance, hair graying, kyphosis, or hyperglycemia (i.e., the symptom or the disease associated with senescence). Furthermore, another aspect of the present disclosure is directed to a method for promoting weight loss or treating type 2 diabetes mellitus in a subject. The aforementioned methods comprise administering to the subject an effective amount of the composition described above, which comprises the (24S)-3β-hydroxy-5α-stigmastan-6-one, and a carrier.

The composition is administered to the subject in the amount of about 0.01 to 1,000 mg/kg body weight of the subject, such as 0.01, 0.02, 0.03, 0.04, 0.05, 0.06, 0.07, 0.08, 0.09, 0.1, 0.2, 0.3 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, or 1,000 mg/kg body weight of the subject; preferably, about 0.1 to 100 mg/kg body weight of the subject. According to some embodiments of the present disclosure, the present composition is administered to the subject in need thereof in the amount of about 1-10 mg/kg. In one working example, the present composition is administered to the subject in the amount of about 5 mg/kg. The dose can be administered in a single aliquot, or alternatively in more than one aliquot. The skilled artisan or clinical practitioner may adjust the dosage or regime in accordance with the physical condition of the patient or the severity of the symptom or the disease associated with senescence.

As would be appreciated, in the case when the subject is afflicted with a disease associated with senescence, the present method may be applied to the subject alone or in combination with an additional therapy (e.g., a surgery, a physical therapy, a physiotherapy, a massage, a phototherapy, or a thermotherapy) or an additional medicament that have some beneficial effects on treating of the symptom or the disease associated with senescence. Depending on the intended therapeutic purposes, the present method may be applied to the subject prior to, in conjunction with, or subsequent to the administration of the additional therapy or the additional medicament.

Said additional medicament may be an anti-hypertensive drug, such as aliskiren, amiloride, amlodipine, atenolol, bendroflumethiazide, bumetanide, candesartan, captopril, clonidine, diltiazem, doxazosin, furosemide, hydralazine, hydrochlorothiazide, labetalol, lisinopril, losartan, methyldopa, metoprolol, minoxidil, nifedipine, nimodipine, phenoxybenzamine, phentolamine, propranolol, ramipril, spironolactone, trimetaphan, valsartan, or verapamil; an anti-inflammatory drug, such as abatacept, acitretin, adalimumab, alefacept, anakinra, anthralin, apremilast, azathioprine, baricitinib, belimumab, benralizumab, betamethasone, bimekizumab, brazikumab, brodalumab, calcipotriene, calcipotriol, calcitriol, canakinumab, certolizumab, clobetasol, coal tar, colchicine, cyclosporine, dapsone, dithranol, dexamethasone, dupilumab, eculizumab, etanercept, fluocinolone, golimumab, guselkumab, hydroxychloroquine, hydrocortisone, infliximab, ixekizumab, lenercept, mepolizumab, methotrexate, methylprednisolone, mirikizumab, mycophenolate mofetil, omalizumab, prednisone, perakizumab, pimecrolimus, remtolumab, reslizumab, rilonacept, risankizumab, rituximab, sarilumab, secukinumab, sulfasalazine, tacrolimus, tazarotene, tildrakizumab, tocilizumab, tofacitinib, tretinoin, upadacitinib, ustekinumab, vedolizumab, or vunakizumab; an anti-osteoporotic drug, such as abaloparatide, alendronate, alfacalcidol, calcitonin, calcium, clodronate, denosumab, eldecalcitol, etidronate, ibandronate, minodronate, monofluorophosphate, pamidronate, parathyroid hormone, raloxifene, risedronate, romosozumab, sodium fluoride (NaF), teriparatide, vitamin D₃, or zoledronic acid; a drug for treating a neurodegenerative disease, such as acyclovir, aducanumab, amantadine, apomorphine, baclofen, carbidopa, dantrolene, donepezil, entacapone, foscarnet, galantamine, levodopa, memantine, penciclovir, pramipexole, rasagiline, riluzole, rivastigmine, ropinirole, selegiline, tacrine, tetrabenazine, tizanidine, or tolcapone.

In the case where the intention is to treat type 2 diabetes mellitus, said additional medicament may be an antidiabetic agent. Non-limiting examples of antidiabetic agents include, without limitation, insulin, metformin, phenformin, buformin, rosiglitazone, pioglitazone, troglitazone, tolimidone, bromocriptine, colesevelam, glitazone, gliptin, tolbutamide, acetohexamide, tolazamide, chlorpropamide, glipizide, glyburide, glibenclamide, glimepiride, gliclazide, glyclopyramide, gliquidone, repaglinide, nateglinide, miglitol, acarbose, voglibose, exenatide, liraglutide, taspoglutide, lixisenatide, semaglutide, dulaglutide, albiglutide, vildagliptin, sitagliptin, saxagliptin, linagliptin, alogliptin, septagliptin, teneligliptin, gemigliptin, dapagliflozin, canagliflozin, empagliflozin, and remogliflozin.

It should be noted that during the term of the present treatment, different therapies or therapeutics may be administered to the subject at different doses, time intervals, via different routes. The doses and time intervals may vary with factors such as described above, and are dependent on the professional considerations of the practitioner; and the routes may be via oral, enteral, buccal, nasal, transdermal, transmucosal, intravenous, intraperitoneal, intraarterial, intracutaneous, subcutaneous, and intramuscular routes.

Basically, the subject treatable by the present method is a mammal; preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### Examples

### Materials and Methods

### 1. Extraction and Isolation of Plumula Nelumbinis (PN)

The dried plumula nelumbinis (2.4 kg) were extracted with ethanol (5 liters) at room temperature for three times, and then with water at 70° C. for 11 times. The combined solutions were vacuum concentrated to give a total crude ethanol extract (523.01 g), which were then subjected to sequentially partitioned between ethyl acetate (236.70 g) and H₂O (286.31 g). The ethyl acetate layer was separated by silica gel chromatography using mixtures of n-hexane and acetone, in which (24S)-3β-hydroxy-5α-stigmastan-6-one (PN6) were eluted in the fraction of dichloromethane:ethyl acetate=49:1.

### 2. Cell Culture

U2OS cells (a human osteosarcoma epithelial cell line; ATCC HTB-96™) and IMR90 cells (a human normal lung fibroblasts; ATCC CCL-186™) were cultured in DMEM supplemented with 10% FBS and 2 mM glutamine. HUVECs were cultured in M199 medium supplemented with 4-(2-hydroxyethyl)-1-piperazineethanesulfonic acid (HEPES), endothelial cell growth supplement (ECGS; Millipore), heparin, NaHCO₃, L-glutamine, sodium pyruvate, and 20% fetal bovine serum (FBS; final concentration). The cells were grown in an atmosphere of 5% CO₂ at 37° C., and passaged to maintain under 90% confluency.

For the population doubling (PD) assay, HUVECs (1×10⁵ cells/3.5 cm dish) were propagated in the presence of DMSO (i.e., control) or PN6 (1 or 2 μM) until senescence (Day 41), and the cell numbers in each passage were recorded to generate a growth curve that represented the cumulative population doubling. The population doubling levels (PDLs) of HUVECs were calculated in every culture to indicate the proliferation capacity of HUVECs under the respective conditions. The population doublings (PDs) were estimated using the equation: PDL=3.32 (log (total viable cells at harvest/total viable cells at seeding). Also, HUVECs at passages PDL4 and PDL11 cultured in 0 μM (i.e., DMSO alone), 1 μM, or 2 μM PN6 were harvested for further analyses (e.g., western blot and SA-β-gal staining).

### 3. Mother Enrichment Program Assay

The MEP strain ZHY2 cells were cultured at 30° C. overnight in liquid yeast 5 extract peptone dextrose (YEPD) medium containing 0, 0.1, 0.5, 2, 10, 25, 50, or 100 μM PN6, and the cultures were diluted to A₆₀₀=0.25 and recovered to the log phase. Cultures were counted and inoculated in culture tubes at a cell density of 2×10³ cells/ml. Estradiol was added to the mixtures to achieve a final concentration of 1 μM, and the mixtures were incubated in a roller drum at 50 rpm and 30° C. for 60 hours. Each group was monitored by harvesting samples at the indicated time points. The collected samples were washed and plated in solid YEPD media without estradiol, and the viability is reported as CFUs per 500 μl. The software OpenCFU 3.8 was used to calculate colonies.

### 4. Micromanipulation Assay

The micromanipulation of the yeast cells was carried out as follows. Prior to analysis, yeast strains BY4741 (WT), BY4741-fob1Δ (fob1Δ), or BY4741-sir2Δfob1Δ (sir2Δfob1Δ) were plated onto fresh solid YEPD medium and grown at 30° C. for two days. Then, individual colonies were picked and grown in solid YEPD media containing: (1) dimethyl sulfoxide (DMSO), (2) 10, 25, or 50 μM PN6, or (3) 25 μM β-sitosterol at 30° C. overnight, and a small number of yeast cells were then plated onto a fresh YEPD plate for lifespan analysis. After overnight growth on the lifespan plates, the cells were arrayed on the plate using a micromanipulator and allowed to grow for approximately 3 hours. Virgin daughter cells were selected and subjected to lifespan analysis. In the lifespan experiments, the plates were incubated at 30° C. during the day and stored at 4° C. overnight. Each experiment consisted of more than 50 mother cells and was independently repeated three times.

### 5. Intracellular NAD⁺ Content

WT yeast cells (BY4741; 4×10¹ per group) were incubated in the presence of DMSO (control) or PN6 (25 μM), or cultured in YEPD medium adjusted to CR 0.1%, for 2 hours, before being harvested. The harvested cells were extracted with 250 μl of 1 M glacial formic acid saturated with butanol, incubated for 30 minutes on ice. Then, 62.5 μl of 100% trichloroacetic acid (TCA) (w/v) was added to each sample, and the samples were incubated on ice for an additional 15 minutes. The samples were pelleted by centrifugation at 17,000×g and 4° C. for 5 minutes, and the supernatant was transferred to another eppendorf tube. The pellets were then washed with 125 μl of 20% TCA, and centrifuged, and the supernatant was combined with the aforementioned supernatant. The combined supernatants were used for the following tests. A series of standard samples were prepared in parallel to create a calibration curve, in which the standard samples having NAD⁺ concentrations at 0, 12.5, 25, 50, 100, 200, 400 μM were prepared in a standard dilution buffer (1 ml 1M glacial formic acid saturated with butanol and 300 μl 20% TCA).

For analysis, each supernatant or standard sample was divided into two sets: the control set and the test set (150 μl per set), each of which was assembled with an alcohol dehydrogenase (ADH) reaction buffer (345 μl, containing 360 mM Tris (pH 9.7), 240 mM lysine, 0.24% (v/v) EtOH). Then, 5 μl of ddH₂O was added into the control set, and 5 μl of 5 mg/ml ADH was added into the test set. After incubation at room temperature for 10 minutes, the absorbance at 340 nm of each sample was measured by a microplate reader. To determine the NAD⁺ levels in each supernatant or standard sample, the absorbance values of the test set (the ADH catalytic set) were divided by the corresponding values of the control set (the water set), which represented the basal NADH levels.

### 6. Acetylation of p53 Via UV Irradiation

U2OS cells (1.2×10⁵ cells/dish) or IMR90 cells (3.0×10⁵ cells/dish) were seeded into a 3.5 cm culture dish, and incubated with 0, 10, 25, 50, or 100 μM PN6 for 16 hours, before subjected to UVB irradiation at 7.5 mJ/m² (for U2OS cells) or 5.0 mJ/m² (for IMR90 cells) for 4 hours. In the case when selisistat (Ex527, an inhibitor for yeast SIR2 or mammalian SIRT1) was added, 1 μl of 50 mM Ex527 was added to the culture one hour prior to the addition of PN6.

### 7. Western Blot Analysis

Cells were harvested with a 1.25× Laemmli buffer and lysed in RIPA lysis buffer containing 1% of Triton X-100 and a protease inhibitor cocktail, and total protein concentrations were measured with a protein assay kit. Proteins in the samples of the lysates with the same amounts of total protein were separated by sodium dodecyl sulfate polyacrylamide gel electrophoresis (SDS-PAGE) (10% polyacrylamide gels) and analyzed by immunoblotting. Primary antibodies against p53ac (K382) (1:1,000), p21 (1:1,000), and 3-actin (1:200,000) were applied to detect the corresponding proteins. Horseradish peroxidase (HRP)-conjugated secondary antibodies against mouse IgG were then incubated with the membranes, and proteins were visualized by chemiluminescence according to the manufacturer's instructions. Protein quantification was performed by software.

### 8. SIR2/SIRT1 Activity Assay

The activity of SIR2/SIRT1 was assessed using a SIRT1 fluorometric drug discovery kit (Enzo Life Sciences, Inc.), in accordance with the manufacturer's instructions. Fluorescence was excited at 360 nm and emitted at 460 nm.

### 9. Senescence-Associated (SA)-β-Gal Staining

HUVECs at 90% confluency were collected, washed, and fixed with 2% formaldehyde and 0.2% glutaraldehyde for 5 minutes. The cells were then subjected to staining by incubation with a staining solution (40 mmol/l citric acid, sodium phosphate, pH 6.0, 1 mg/ml 5-bromo-4-chloro-3-isolyl-β-D-galactoside (X-gal, Sigma), 5 mmol/l potassium ferrocyanide, 5 mmol/l potassium ferricyanide, 150 mmol/l NaCl, and 2 mmol/l MgCl₂) at 37° C. for 18-24 hours. The results of SA-β-gal-positive cells were observed by microscopy, and were counted for over 300 cells in at least three independent fields.

### 10. Animals

All animal procedures were conducted under protocols (Protocol #CGU108-134) that complied with relevant regulations and approved by Research Guidelines for the Care and Use of Laboratory Animals, Chang Guan University. Male C57BL/6J male mice (at 34 weeks of age) obtained from National Laboratory Animal Center (NLAC) were maintained at 23±1° C. and at humidity of 45-70% with a 12 hour dark/light cycle (7:00 am/pm lights on/off). After reaching 50 weeks of age, the mice were randomly divided into two groups: Group I (SD), fed a standard rodent chow (n=27); and Group II (PN6), fed a standard rodent chow containing 50 mg/kg PN6 (n=10). Animals had ad libitum access to food and water throughout the study (the mice were sacrificed at 131 weeks of age), during which their body weight and food intake were measured weekly.

### 11. Rearing Behavior

The rearing behavior of the male C57BL/6J mice fed a standard rodent chow with or without 50 mg/kg PN6 for 22 weeks (at 72 weeks of age; mid-term) or for 61 weeks (at 111 weeks of age; long-term) was analyzed using the OxyletPro System, in which the mice were housed individually in metabolic cages that were equipped with infrared light beam frames surrounding them. Mid-term: SD, n=16; PN6, n=8; long-term: SD, n=12; PN6, n=8.

### 12. Grip Strength Test

The grip strength of the male C57BL/6J mice was measured at 87 weeks (mid-term) and 111 weeks (long-term) of age using an adjustable metallic grid mounted with a force sensor (DTG-5, DigiTech, JP). To conduct the test, the mice were placed on the aforementioned test device and were held gently by the tail, allowing them to grip the grid with forelimbs, hindlimbs, or four limbs (for measuring 2-paw and 4-paw grip strength, respectively). The device automatically measured and recorded the peak force in grams (g). Five trials were performed for each test, and the mean values were used to determine the grip strength of each mouse. Mid-term: SD, n=30; PN6, n=8; long-term: SD, n=23; PN6, n=8.

### 13. Rotarod Test

The male C57BL/6J mice were subjected to a rotarod test at 88 weeks (mid-term) and 112 weeks (long-term) of age. The mice underwent a 3-day habituation period, during which they were placed on the rotarod at a constant speed (4 rpm), and were required to remain on the rotarod for 1 minute at a time every day. To conduct the actual test, each mouse was subjected to five trials, with a 10-15 minute rest period between each trial. For each trial, the tests for the constant and accelerated speeds were conducted sequentially according to the following schedule: During a 5-minute trial period, a run consisted of an initial constant rotating speed of 4 rpm and a subsequent accelerated rotating speed accelerated from 4 to 40 rpm was performed 5 times. Trials were concluded if the animals fell off the rod, showed passive cycling, or when the designated timing had elapsed. The latency to falling was recorded, and the average latency to falling was calculated for each trial. Data are presented as the average of the 5 trials. Mid-term: SD, n=30; PN6, n=8; long-term: SD, n=22; PN6, n=8.

### 14. Intraperitoneal Insulin Tolerance Test (IpITT)

The male C57BL/6J mice, aged 107 weeks, were fasted for 16 hours while having free access to water, and their body weights were measured before the test started. The test was conducted by fasting the mice for 5 hours, and then injecting them with 1.25 U/kg of human recombinant insulin. Blood droplets were collected from the tail vein just prior to the insulin injection (time 0; baseline), and at 15, 30, 60, and 90 minutes following the injection. Blood glucose levels were measured using a blood glucose meter (Accu-Chek Guide; Roche, DE).

### 15. Metabolic Breath Analysis Test

The metabolic breath analysis test was carryout out using the OxyletPro System. The male C57BL/6J mice fed a standard rodent chow with or without 50 mg/kg PN6 for 22 weeks (at 72 weeks of age; mid-term) were placed in the metabolic cages of the OxyletPro System for a 24-hour habituation period before actual recordings and remained in the cages for the following 48 hours. The OxyletPro System was equipped with an air supply and switching unit that provided independent flow control to the cage and collected the products produced therein. The gas analyzer was connected in tandem to the OxyletPro System for O₂ and CO₂ concentration analyses, from which the oxygen consumption (VO₂), the carbon dioxide production (VCO₂), and the energy expenditure (EE) were automatically calculated. SD, n=12; PN6, n=7.

### 16. Statistics and Reproducibility

Graphic visualization and statistical analyses were performed by GraphPad Prism 6. All values are exhibited as the mean S.D., and the exact p value was provided in figures or legends. For the experiments involving the SA-β-gal staining, and all the animal experiments, statistical significance was determined using an unpaired Student's t-test. For the experiments involving the MEP assay, the RLS assessment, the intracellular NAD⁺ measurement, the western blotting quantification results, the SIRT1 activity assay, and the PDL of HUVECs, statistical significance was determined using one-way ANOVA with multiple comparisons. For the survival curve of the mouse model, statistical significance was determined using the Gehan-Breslow-Wilcoxon test. Each experiment was performed at least three times as independent replicates.

### Example 1 Isolation and Identification of PN6

The PN6 was extracted and purified from plumula nelumbinis (PN) in accordance with the procedures described in the section of “Materials and Methods.” The identities of the purified PN6 (i.e., (24S)-3β-hydroxy-5α-stigmastan-6-one) was confirmed by NMR proton spectroscopy.

### Example 2 PN6 Extended Replicative Lifespan (RLS) of Yeasts

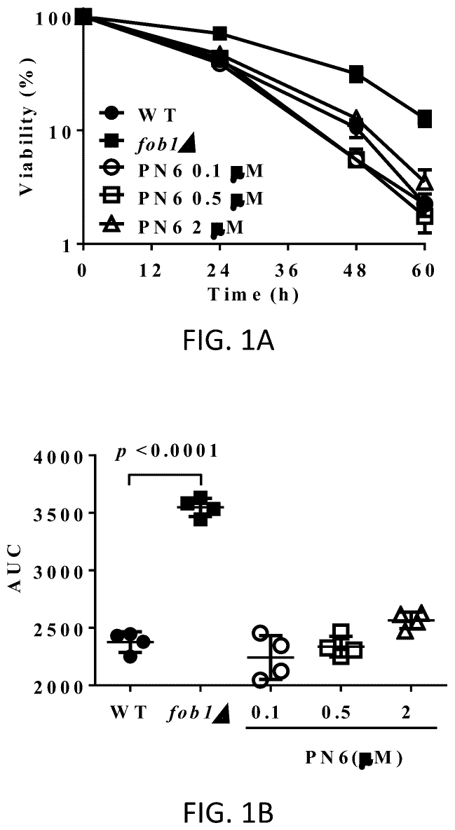

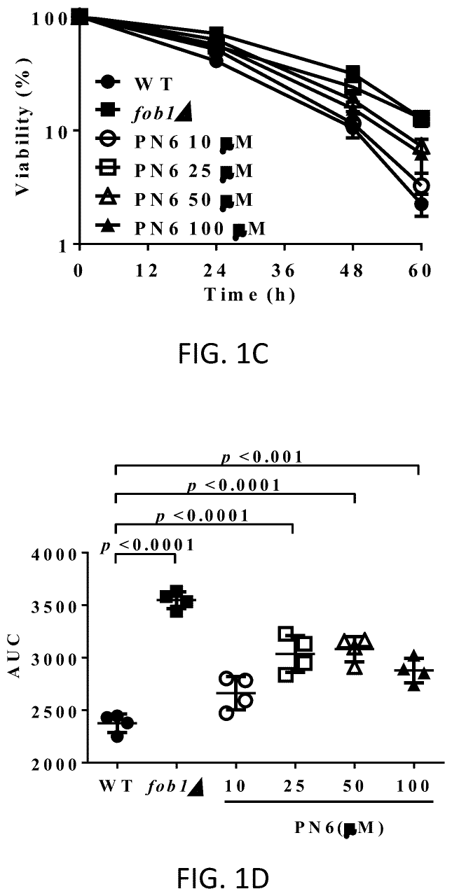

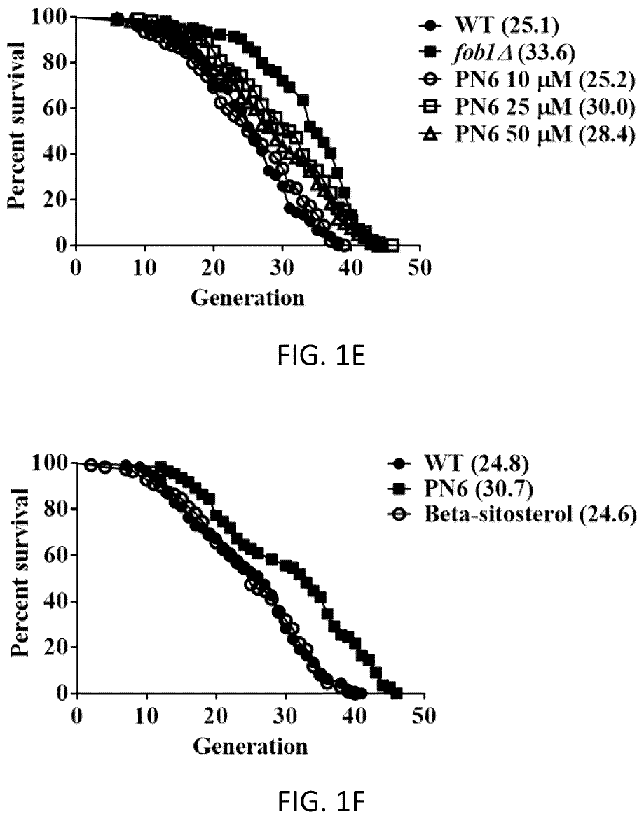

The effect of PN6 of Example 1 on extending the RLS of yeasts was investigated by using the MEP assay. Based on the results shown in FIGS. 1A-1D, the viability of yeast ZHY2 increased proportionally with the concentration of PN6, particularly when the concentration of PN6 exceeded 10 μM. These findings suggest a clear dose-dependent relationship between PN6 and the extension of yeast RLS, indicating that PN6 is capable of extending the RLS of yeast. Further, the ability of PN6 in extending the RLS of yeast was confirmed by the miromanipulation assay, in which PN6 at concentrations of 25 and 50 μM significantly extended the RLS of yeast by an average of 4.9 (14.3%) and 3.3 (11%) generations, respectively (FIG. 1E). The extension of the RLS of yeast was not seen when yeast was treated with 3-sitosterol (FIG. 1F), reflecting that the unique ability of PN6 to prolong the RLS of yeast lies in its chemical structure, particularly the functional groups present on the steroid core structure of PN6. Collectively, these results evidenced that PN6 has the capability to extend the RLS of yeast, and exhibits good lifespan-extending activity even at lower concentrations.

### Example 3 PN6 Promoted Yeast RLS in SIR2-Dependent Manner

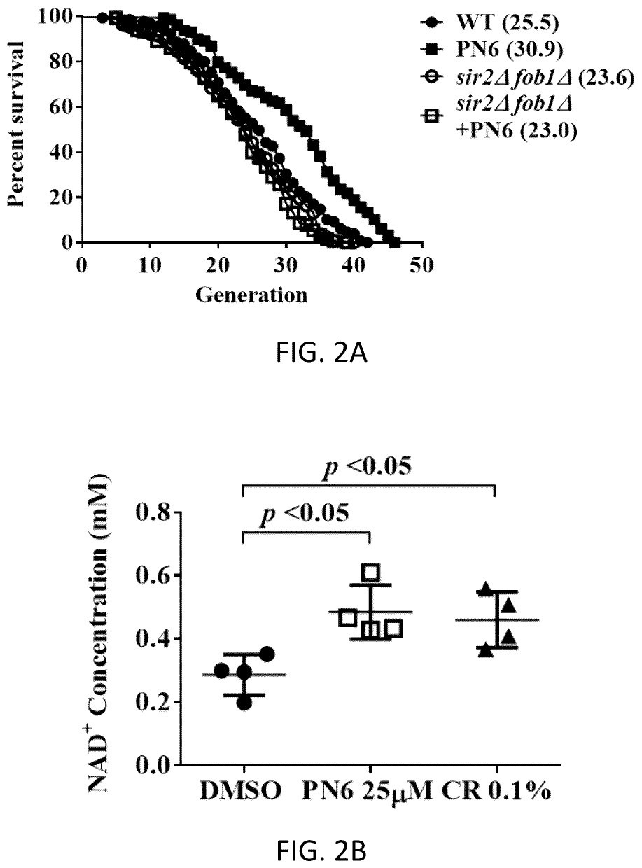

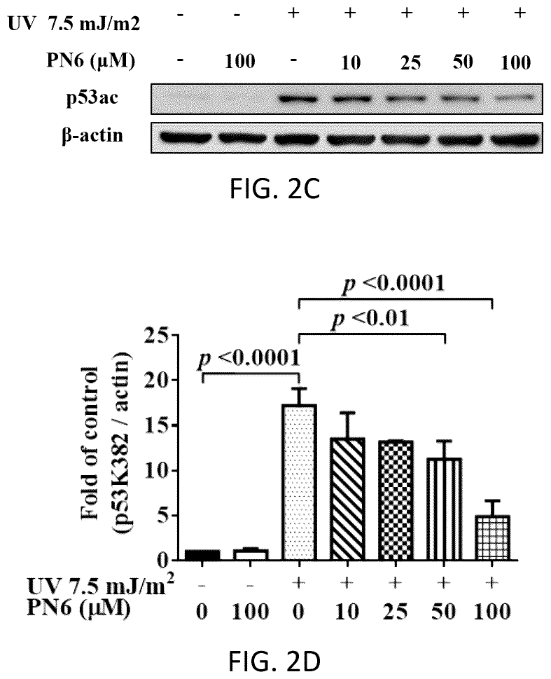

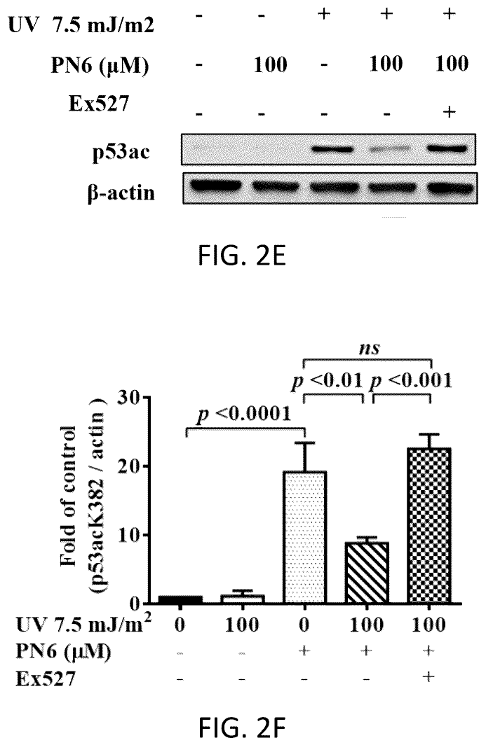

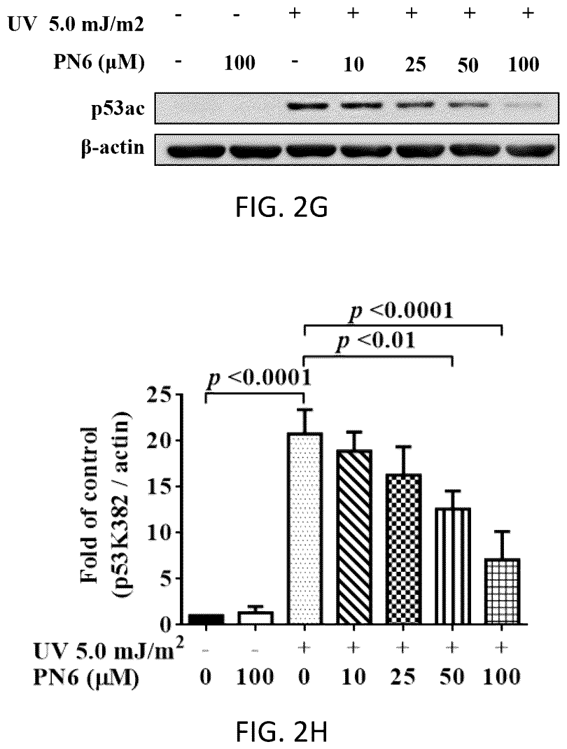

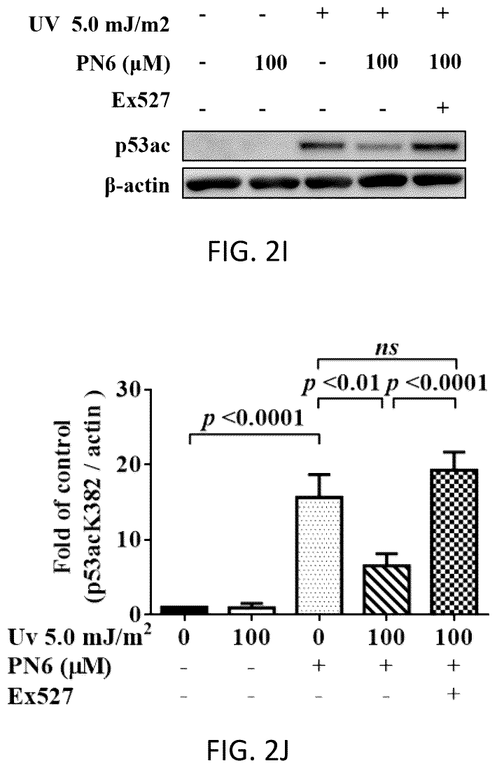

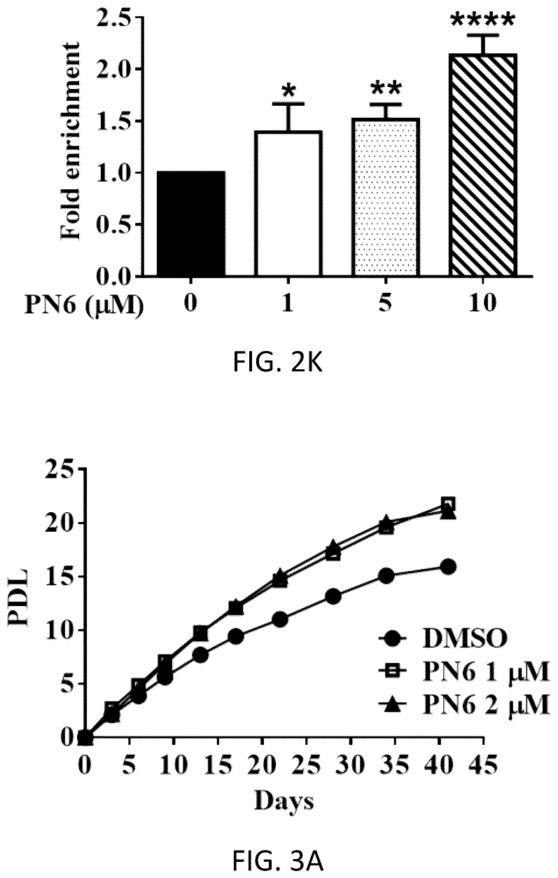

The mechanism underlying the lifespan-extending effect of PN6 was investigated in this example. There are two key regulators SIR2 and TOR1 in regulating the lifespan of yeast, in which SIR2, a protein known for its lifespan-extending properties, plays a crucial role in regulating the replicative lifespan of yeast. To determine whether the genetic pathway of SIR2 is involved in the lifespan-extending effect of PN6, the sir2Δfob1Δ double deletion strain of yeast was employed and was cultured in the presence or absence of PN6. According to the results presented in FIG. 2A, PN6 was unable to extend the lifespan of the sir2Δfob1Δ mutant strain of yeast, suggesting that the ability of PN6 to promote yeast RLS was compromised by the loss of SIR2. In addition, whether the genetic pathway of TOR1 plays a role in the lifespan-extending effect of PN6 was also investigated. To this purpose, the tor1Δ deletion strain of yeast was used and was cultured in the presence or absence of PN6. Nevertheless, the ability of PN6 to increase yeast RLS was not affected by the deletion of the TOR1 gene, indicating that PN6 increases yeast RLS through a TOR-independent pathway (data not shown). Taken together, these results evidenced that PN6 increases RLS via a SIR2-dependent pathway.

It has been reported that the biological function of yeast SIR2 (or mammalian homolog, SIRT1) is derived from its increased activity rather than an increase in protein expression, thus, the effect of PN6 on the activity of SIR2 in yeast was investigated. To this end, NAD⁺ content in the cells was measured as an indicator of the SIR2 activity therein, as NAD⁺ was found to positively correlated with the SIR2 activity. Caloric restriction (CR) is known to increase the intracellular NAD⁺ content and thus enhances Sir2 activity, and was used as a positive control (CR 0.1%) in this experiment. As shown in FIG. 2B, treatment with 25 μM PN6 significantly increased the NAD⁺ content of yeast, similar to the positive control group (CR 0.1%). Accordingly, PN6 increases NAD⁺ content, indicating its function as an NAD⁺ activator. Collectively, these results clearly indicated that PN6 increased the RLS of yeast by enhancing its SIR2 activity.

The ability of PN6 to enhance the SIRT1 activity in the mammalian cell system was also determined. It is known that P53 protein in cells is acetylated at its K382 site by UV irradiation, which can be specifically removed by SIRT1; accordingly, the level of acetylation of p53 was used as an indicator in this example for evaluating the effect of PN6 on SIRT1 activity. As shown in FIGS. 2C-2D, UV irradiation caused significant acetylation of P53 at K382 in U2OS cells, and the degree of acetylation decreased in a dose-dependent manner with increasing concentrations of PN6. The SIRT1 activity induced by PN6 was compromised by the addition of a SIRT1 inhibitor, Ex527, which further confirmed that PN6 indeed increased the SIRT1 activity (FIGS. 2E-2F). Similar results were observed in IMR90 cells, where PN6 increased SIRT1 activity to reverse acetylation of P53 at K382 (FIGS. 2G-2H). However, this effect was eliminated in the presence of Ex527, as shown in FIGS. 2I-2J. The SIRT1 activity in PN6-treated cells was evaluated using a SIRT1 activity assay kit, further confirming that PN6 may directly increase the SIRT1 activity (FIG. 2K). These results confirmed that PN6 enhanced the activity of yeast SIR2 or mammalian SIRT1, suggesting its potential function as an activator for both, and ultimately leading to an increased RLS in yeast.

### Example 4 PN6 Ameliorated Cellular Senescence

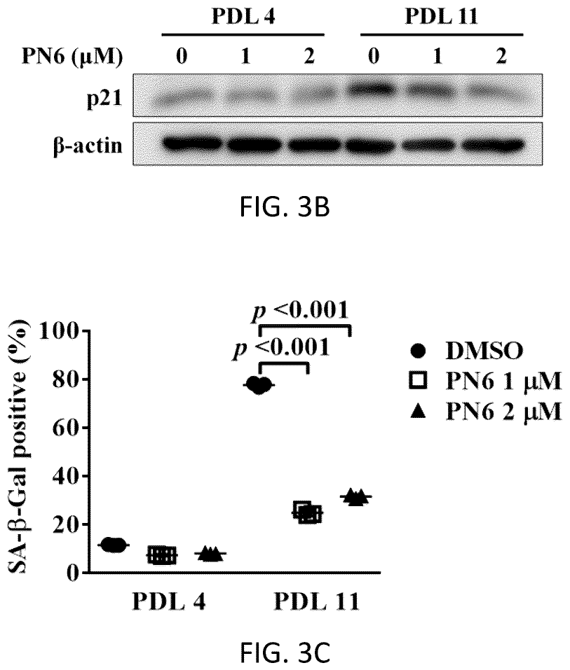

In this example, the effect of PN6 on mammalian cellular senescence was investigated, by monitoring the population doubling level (PDL). It was found that PN6 could increase the PDL of HUVECs (FIG. 3A), suggesting that PN6 could ameliorate cellular senescence.

According to the literature, p21 and SA-β-gal are representative markers of replicative exhaustion characteristics in mammalian cells. To this end, the effect of PN6 on the expression of p21 and SA-β-gal were explored. Reference is made to FIG. 3B, which indicated that in the late stage of HUVECs (i.e., HUVECs at PDL11), rather than in the early-middle stage of HUVECs (i.e., HUVECs at PDL4), the expression level of p21 increased in the absence of PN6, suggesting cell cycle arrest, whereas the p21 expression level decreased dose-dependently with the presence of PN6 in HUVECs at PDL11. Further, PN6 also resulted in a decrease in the number of the SA-β-gal-positive senescent cells in the late stage of HUVECs (i.e., HUVECs at PDL11), as compared with that in the control group without PN6 (FIG. 3C). Taken together, these data revealed that PN6 could ameliorate cellular senescence in mammalian cells.

### Example 5 PN6 Delayed Aging Process in Mice

### 5.1 Survival Rate of the Aged Mice

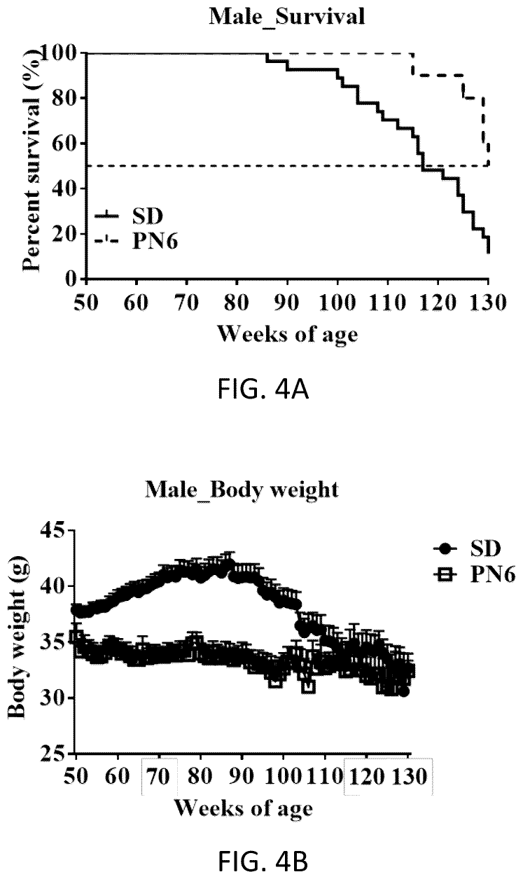

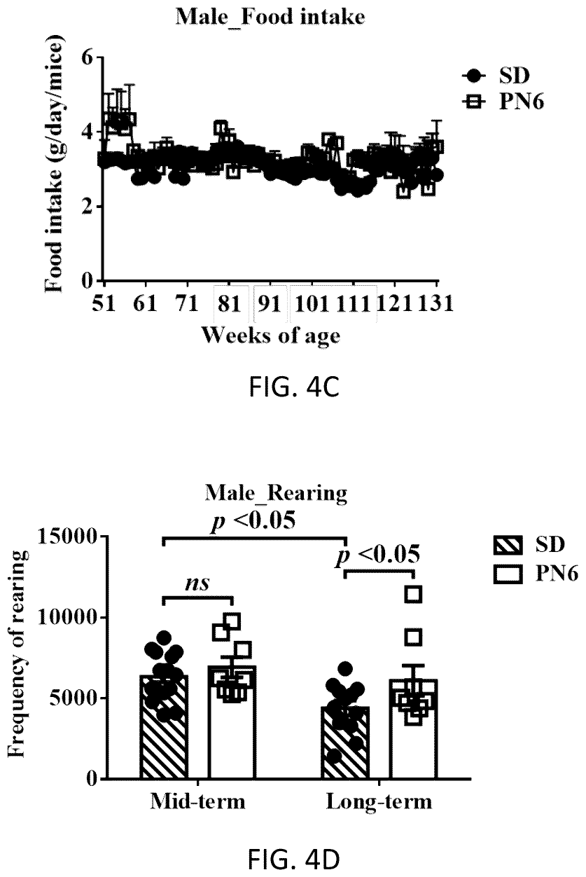

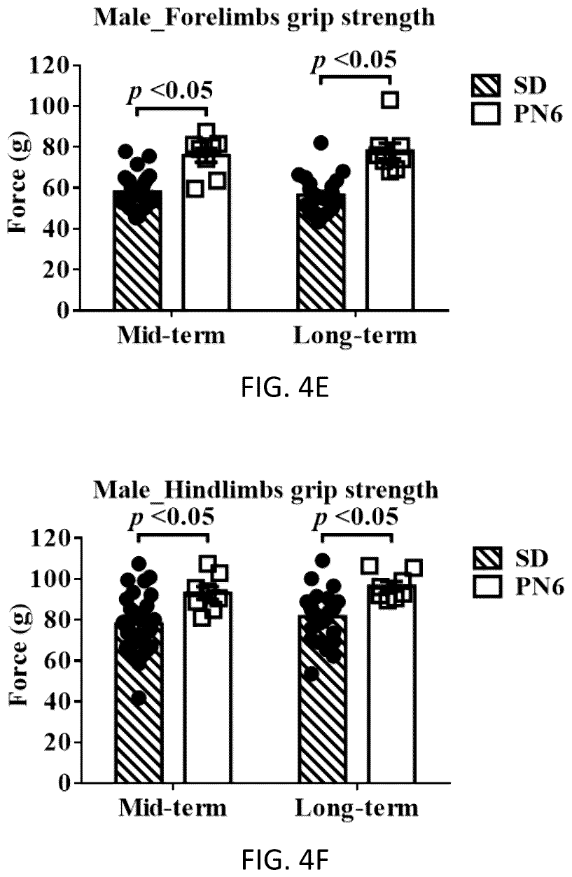

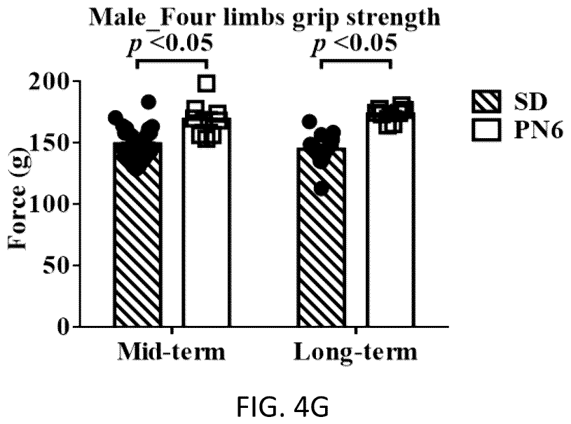

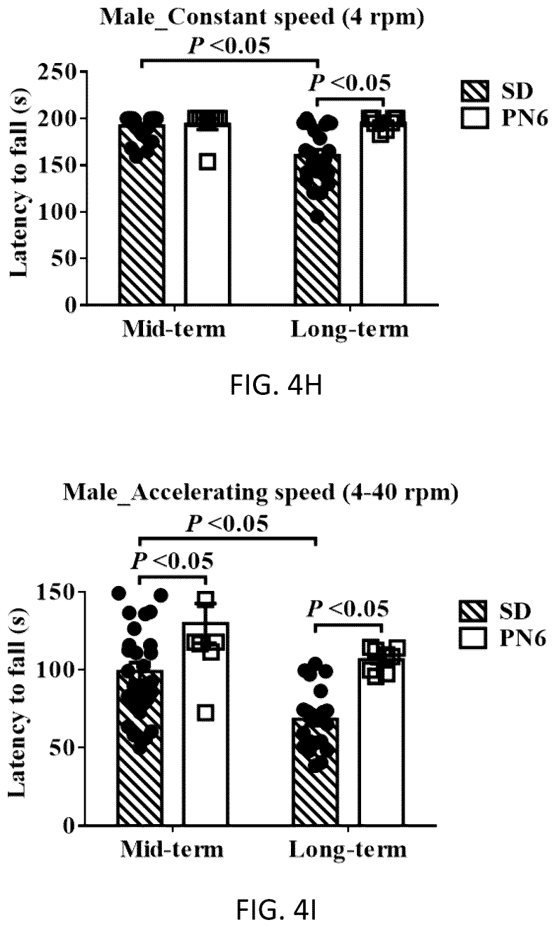

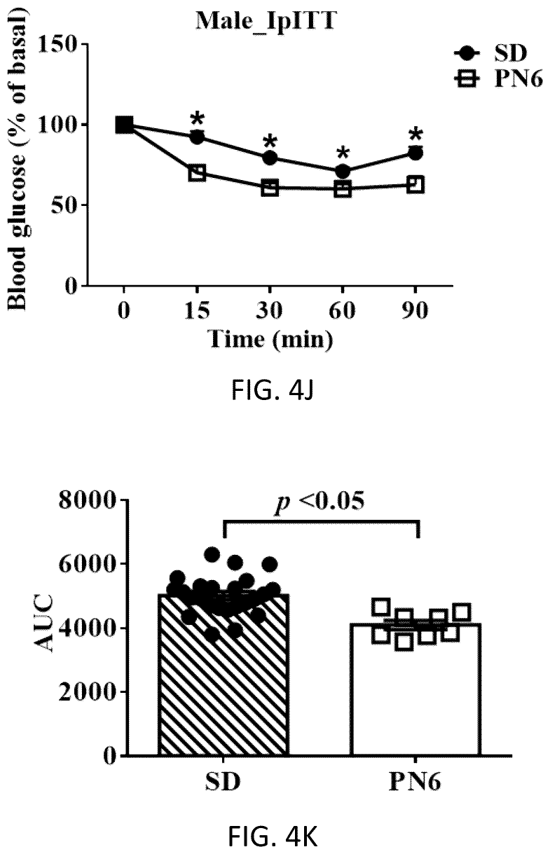

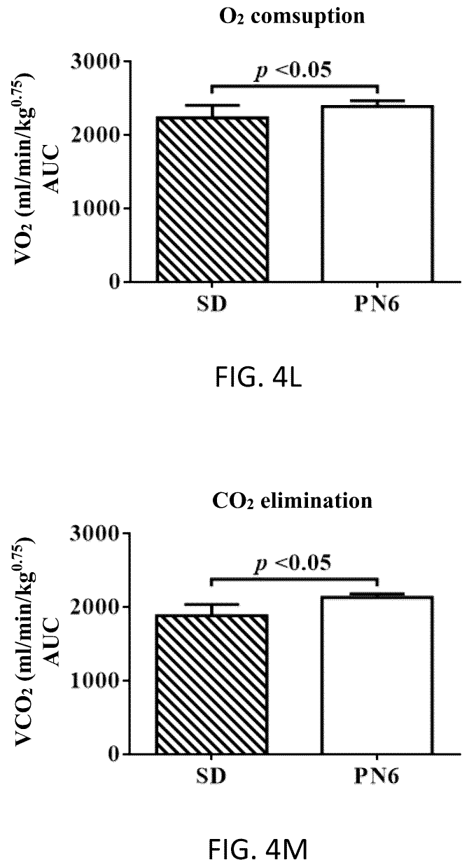

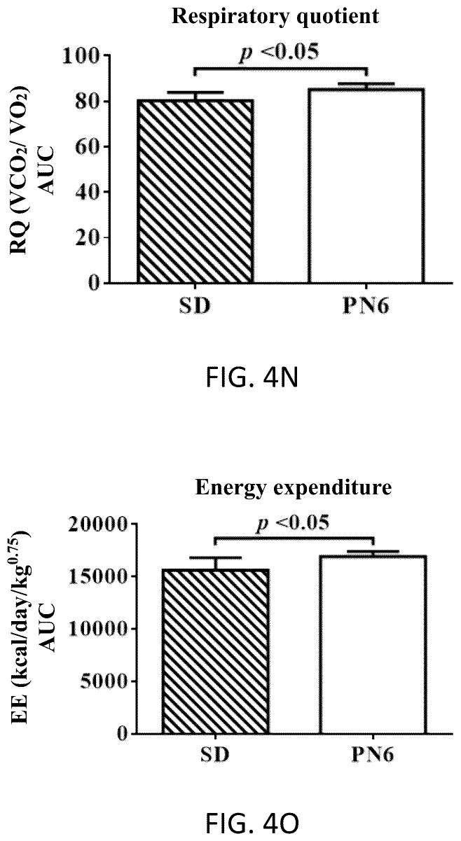

The ability of PN6 in delaying aging process in mice was examined in the present example. To this end, aged C57BL/6J mice (fifty-week-old) were fed a standard rodent chow with or without PN6 ad libitum until they were sacrificed at 131 weeks of age. Results are shown in FIGS. 4A-4C.

The results shown in FIG. 4A clearly evidenced the long-term benefits of PN6 treatment, as demonstrated by the significantly extended survival curves observed in the mice of the PN6 group. The results confirmed that the mice in the PN6 group overall exhibited higher survival rate as compared with that of the mice in the SD group. At the conclusion of the study (at 130 weeks of age), 50% of the mice in the PN6 group remained alive, while less than 10% of the mice in the SD group had survived. The results evidenced that PN6 significantly increased the lifespan of the aged mice. Note that the trajectories of the body weight did not ultimately differ significantly between the two treatment groups, although the mice in the SD group exhibited rapid weight gain during the early-middle stage of the experiment (as shown in FIG. 4B). The trajectories of body weight in the PN6 group remained in a steady state, which reflects the fact that PN6 assists individuals in controlling their body weight (FIG. 4B). This substantiates the potential of PN6 to promote weight loss in individuals. It was also found that during the entire course of the experiment, the food intake did not diverge between the two treatment groups (FIG. 4C), suggesting that the observed increase in lifespan can be attributed to PN6 treatment rather than a reduction in caloric intake.

### 5.2 Visible Changes in Appearance of the Aged Mice

The visible changes in appearance of the mice in the two treatment groups were also recorded and summarized in Table 1. These data suggested that PN6 supplementation could delay the aging process which led to prolonging longevity in aged mice, as well as improving the visible appearance to a youthful phenotype.

TABLE 1
Visible changes in appearance of the
mice in the two treatment groups

| Item | The SD group | The PN6 group |
|---|---|---|
| Hair graying | 70% | 25% |
| Muscle weakness | 48% | 13% |
| Kyphosis | 30% | 13% |

### 5.3 Physical Changes of the Aged Mice

This present example aimed to address the beneficial effects of PN6 on age-associated physical dysregulations by evaluating through measurements of the rearing behavior, the grip strength, and performance on the rotarod tests. Results are provided in FIGS. 4D-4I.

The rearing behavior includes standing and raising their heads, which are rodent species-typical behaviors in exploring the surroundings in a new environment. It was found that the old-aged mice exhibited an overall reduction in exploration of their surroundings, as compared to the middle-aged mice. In contrast, long-term PN6 treatment was found to increase the exploratory activity in the old-aged mice, as shown in FIG. 4D. The grip strength is a suitable measure of muscle strength and can serve as an indicator of health and physical function, and the grip strength of the forelimbs, hindlimbs, and all four limbs of the mice was assessed in both the PN6 and SD groups, both in the mid-term and long-term. As compared to the SD group, the mice in the PN6 group exhibited increased grip strength in their forelimbs, hindlimbs, and all four limbs both in the mid-term and long-term, as illustrated in FIGS. 4E-4G. Given that age-related skeletal muscle degeneration, which can cause muscle atrophy and reduced performance, often leads to exercise intolerance, the rotarod test was used to evaluate the physical function of balance and motor coordination in the mice from two treatment groups both in the mid-term and long-term periods. According to FIGS. 4H-4I, the motor skills (i.e., having longer latency to falling) of the PN6-fed mice steadily improved both in the mid-term and long-term periods, either in rotarod at constant speed or at accelerating speed, as compared with the SD-fed mice.

Taken together, the results indicated that PN6 may ameliorate the age-related decline in terms of physical functions.

### 5.4 Blood Sugar Homeostasis of the Aged Mice

In this example, the effect of PN6 in the old-aged mice was investigated via monitoring parameters related to blood sugar homeostasis, including hyperglycemia, glucose tolerance, and insulin resistance. The IpITT test revealed that the mice fed with PN6 had a significantly higher insulin sensitivity compared to that of the mice fed with SD (FIGS. 4J-4K). Nevertheless, no significant differences in glucose tolerance were observed between the two treatment groups of mice (data not shown). Accordingly, these data suggest that PN6 has the potential to be used in the treatment of diseases associated with hyperglycemia, such as type 2 diabetes mellitus.

### 5.5 Metabolic Alteration of the Aged Mice

In this example, the metabolic alteration in the aged mice treated with or without PN6 were assessed by monitoring two respiratory parameters, namely oxygen (O₂) consumption and carbon dioxide (CO₂) elimination, which are indicative of alterations in basal metabolic rate that occur during aging. Based on the results presented in FIGS. 4L-4O, mice fed with PN6 had significantly higher rates of O₂ consumption and CO₂ elimination compared to those fed with SD (FIGS. 4L-4M), resulting in higher respiratory quotient and energy expenditure (FIGS. 4N-4O). Taken together, these data demonstrated that PN6 may enhance the basal metabolic rate of the aged mice.

In sum, the data disclosed herein demonstrated that the (24S)-3β-hydroxy-5α-stigmastan-6-one (PN6) possessed anti-aging properties reflected in many aspects, and thus may serve as a potential therapeutic agent for the development of a medicament for treating symptoms or diseases associated with senescence.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for extending lifespan and/or delaying aging in a subject comprising administering to the subject an effective amount of (24S)-3β-hydroxy-5α-stigmastan-6-one, wherein the delaying aging comprises increasing vitality, muscle strength or motor coordination, insulin sensitivity, or basal metabolic rate; and/or reducing muscle weakness, loss of balance, hair graying, kyphosis, or hyperglycemia.

2. The method of claim 1, wherein the (24S)-3β-hydroxy-5α-stigmastan-6-one is an activator of mammalian SIRT1 or nicotinamide adenine dinucleotide (NAD⁺).

3. The method of claim 1, wherein the (24S)-3β-hydroxy-5α-stigmastan-6-one is administered in the amount of about 1-10 mg/kg.

4. The method of claim 1, wherein the subject is a human.

5. A method for promoting weight loss in a subject comprising administering to the subject an effective amount of (24S)-3β-hydroxy-5α-stigmastan-6-one.

6. The method of claim 5, wherein the (24S)-3β-hydroxy-5α-stigmastan-6-one is administered in the amount of about 1-10 mg/kg.

7. The method of claim 5, wherein the subject is a human.

8. A method for treating type 2 diabetes mellitus in a subject comprising administering to the subject an effective amount of (24S)-3β-hydroxy-5α-stigmastan-6-one.

9. The method of claim 8, wherein the (24S)-3β-hydroxy-5α-stigmastan-6-one is administered in the amount of about 1-10 mg/kg.

10. The method of claim 8, further comprising administering an antidiabetic agent to the subject.

11. The method of claim 10, wherein the antidiabetic agent is selected from the group consisting of insulin, metformin, phenformin, buformin, rosiglitazone, pioglitazone, troglitazone, tolimidone, bromocriptine, colesevelam, glitazone, gliptin, tolbutamide, acetohexamide, tolazamide, chlorpropamide, glipizide, glyburide, glibenclamide, glimepiride, gliclazide, glyclopyramide, gliquidone, repaglinide, nateglinide, miglitol, acarbose, voglibose, exenatide, liraglutide, taspoglutide, lixisenatide, semaglutide, dulaglutide, albiglutide, vildagliptin, sitagliptin, saxagliptin, linagliptin, alogliptin, septagliptin, teneligliptin, gemigliptin, dapagliflozin, canagliflozin, empagliflozin, and remogliflozin.

12. The method of claim 8, wherein the subject is a human.
